(19) United States
(12) Patent Application Publication
(10) Pub. No.: US 2026/0237524 A1
(43) Pub. Date: Aug. 13, 2026

(54) TECHNIQUES FOR INCREASING COMPUTATIONAL EFFICIENCY OF ANALYZING CLINICAL INTERVENTION DATA WHILE CONSIDERING PATIENT TREATMENT OUTCOME PREFERENCES

(71) Applicant: INTERNATIONAL DRUG DEVELOPMENT INSTITUTE, S.A., Ottignies-Louvain-la-Neuve (BE)

(72) Inventors: Marc Eric Georges Raymond BUYSE, Genval (BE); Tomasz BURZYKOWSKI, Hasselt (BE); Jean-Christophe CHIEM, Brussels (BE); Everardo DELFORGE SAAD, Brussels (BE)

(73) Assignee: INTERNATIONAL DRUG DEVELOPMENT INSTITUTE, S.A., Ottignies-Louvain-la-Neuve (BE)

(21) Appl. No.: 19/635,183

(22) Filed: Mar. 31, 2026

Related U.S. Application Data
(63) Continuation of application No. 18/742,369, filed on Jun. 13, 2024, now abandoned.
Application No. 18/742,369 is a continuation of application No. 18/653,133, filed on May 2, 2024, now abandoned.
Application No. 18/653,133 is a continuation of application No. PCT/IB2022/000661, filed on Nov. 2, 2022.
(60) Provisional application No. 63/275,137, filed on Nov. 3, 2021.

(51) Int. Cl. G16H 50/70; G16H 10/20; G16H 10/60

(52) U.S. Cl. CPC G16H 50/70; G16H 10/20; G16H 10/60

(57) ABSTRACT

Techniques for increasing computational efficiency of analyzing clinical intervention data while considering patient treatment outcome preferences are provided. A ranked priority of a plurality of treatment outcomes is received from a patient. Pairwise comparisons based on a highest-ranked treatment outcome for pairs of experimental subjects and control subjects are performed to assign each pair a label. For each pair assigned a tie label but not pairs assigned a win label or a loss label, a pairwise comparison based on a next-highest ranked treatment outcome is performed to attempt to replace the tie label with the win label or the loss label, thereby avoiding the computational cost of comparing all of the pairs based on the next-highest ranked treatment outcome. In response to determining that a net treatment benefit indicates that the clinical intervention should be administered, the clinical intervention is caused to be administered to the patient.

### CROSS-REFERENCE(S) TO RELATED APPLICATION(S)

This application is continuation of U.S. Application No. 18/742,369, filed June 13, 2024, which is a continuation of U.S. Application No. 18/653,133, filed May 2, 2024, now abandoned, which is a continuation of International Application No. PCT/IB2022/000661, filed November 2, 2022, which claims the benefit of U.S. Provisional Application No. 63/275,137, filed November 3, 2021, each of which is incorporated by reference herein in its entirety.

### BACKGROUND

Statistical analysis of clinical data may be performed to identify treatment plans for subjects (e.g., patients in clinical trials) and to evaluate net benefits of treatment plans (e.g., net treatment benefits).

### SUMMARY

Currently, there may be a lack of flexible approaches to identifying and evaluating net benefits of treatment plans (e.g., net treatment benefits). Thus, there exists a need for improved methods and systems which takes into account personalized preferences. In some embodiments, methods of the present disclosure may comprise performing assessments at the patient-level or individual-level (e.g., using patient-level treatment outcomes and data).

The present disclosure provides improved methods and systems for carrying out statistical analysis on clinical data, which may allow the consideration of a plurality of outcome interests. Inputs from a user, such as a clinician, a medical professional, or a patient may be incorporated into the statistical analysis so that statistical inferences over the underlying clinical data may be made with granularity. For example, statistical inferences may be made on a case-by-case scenario based on a user's preferences, needs, wants, or a combination thereof.

In an aspect, the present disclosure provides a computer-implemented method for identifying a net treatment benefit of a clinical intervention for a subject, comprising: (a) obtaining a dataset for a treatment set of subjects and a reference set of subjects, wherein the treatment set of subjects receives the clinical intervention, and wherein the reference set of subjects does not receive the clinical intervention; (b) obtaining a plurality of treatment outcomes for the treatment set of subjects and the reference set of subjects; (c) receiving user input of a prioritization function of the plurality of treatment outcomes; (d) performing a set of pairwise comparisons between a first subject selected from the treatment set of subjects and a second subject selected from the reference set of subjects, at least in part by performing a prioritized comparison of the plurality of treatment outcomes between the first subject and the second subject based at least in part on the prioritization function received in (c); and (e) determining the net treatment benefit of the clinical intervention for the subject, based at least in part on the set of pairwise comparisons performed.

In some embodiments, the clinical intervention comprises an intervention that may be compared between a case group and a control group.

In some embodiments, the clinical intervention is selected from the group consisting of a medication, a cell-based or gene therapy, a drug treatment, a medical device, a surgical intervention, a radiotherapy, radioisotopic/nuclear therapy, physical therapy, occupational therapy, phonoaudiological therapy, a rehabilitation intervention, a psychological intervention, an immunotherapy, a digital health intervention, and a behavioral intervention. In some embodiments, the clinical intervention comprises the drug treatment. In some embodiments, the drug treatment comprises an approved drug treatment. In some embodiments, the drug treatment comprises an experimental drug treatment.

In some embodiments, the treatment outcomes are measured by discrete variables, continuous variables, ordinal variables, or time-to-event variables. In some embodiments, the treatment outcomes are measured by discrete variables. In some embodiments, the treatment outcomes are measured by continuous variables. In some embodiments, the treatment outcomes are measured by ordinal variables. In some embodiments, the treatment outcomes are measured by time to-event variables.

In some embodiments, the treatment outcomes comprise a member selected from the group consisting of event-free survival time, progression-free survival time, overall survival time, another time to event, efficacy, safety, quality of life, a score (functional score, performance score, toxicity grade, behavioral score, a composite score, an index score, or a combination thereof), and a biomarker (chemical, genomic, epigenomic, gene expression, protein, metabolite, clinical test result corresponding to a disease).

In some embodiments, the first subject is randomly selected from the treatment set of subjects, and wherein the second subject is randomly selected from the reference set of subjects.

In some embodiments, (e) comprises determining a difference in the treatment outcomes between the first subject and the second subject. In some embodiments, (e) comprises comparing the difference in the treatment outcomes between the first subject and the second subject to a clinical threshold. In some embodiments, (e) comprises comparing each of a plurality of treatment outcomes between the first subject and the second subject.

In some embodiments, the method further comprises characterizing a pairwise comparison as a win, a loss, a tie, or an indeterminate comparison based at least in part on the difference in the treatment outcomes between the first subject and the second subject. In some embodiments, a pairwise comparison is characterized as a win, a loss, or a tie based at least in part on the difference in the treatment outcomes being a positive difference greater than a threshold, a negative difference greater than a threshold, or a difference less than a threshold, respectively.

In some embodiments, the method further comprises determining a likelihood that the first subject has a better treatment outcome than the second subject, based at least in part on the set of pairwise comparisons. In some embodiments, the likelihood comprises a probability or a net probability.

In some embodiments, (d) comprises comparing the treatment outcomes between the first subject and the second subject at least in part by comparing a net benefit minus a net harm between the first subject and the second subject. In some embodiments, the net benefit comprises a member selected from the group consisting of event-free survival time, progression-free survival time, overall survival time, another time to event, efficacy, safety, quality of life, a score (functional score, performance score, toxicity grade, behavioral score, a composite score, an index score, or a combination thereof), and a biomarker (chemical, genomic, epigenomic, gene expression, protein, metabolite, clinical test result corresponding to a disease). In some embodiments, the net harm comprises an adverse event grade selected from the group consisting of a side effect and a toxicity of the clinical intervention.

In some embodiments, the treatment set of subjects and the reference set of subjects comprise subjects having a disease or disorder. In some embodiments, the disease or disorder is selected from the group consisting of allergic, articular, bone, cardiac, dermatologic, endocrinologic, gastrointestinal, gynecologic, hematologic, immunologic, infectious, neurologic, ophthalmic, otolaryngologic, pulmonary, psychiatric, renal, rheumatologic, urinary, and vascular disorders, as well as benign and malignant tumors, inborn errors of metabolism, obstetric conditions, and trauma, cancer, CVD, diabetes, and ophthalmic diseases. In some embodiments, the disease or disorder comprises cancer.

In some embodiments, the treatment outcomes are obtained by performing a biomarker test on the treatment set of subjects and the reference set of subjects. In some embodiments, the biomarker test comprises a laboratory test selected from the group consisting of biochemistry, hematology, coagulation, microbiology, molecular genetics, cytogenetics, flow cytometry, and pathology, imaging and radiology (X-rays, fluoroscopy, computed tomography, magnetic resonance imaging, ultrasound, echocardiography, positron-emission tomography, single-photon emission tomography, radionuclide imaging, optic coherence tomography, electrocardiography, electroencephalography, electromyography, evoked potential, audiometry, visual acuity testing, visual field testing, slit-lamp examination), and diagnostic, prognostic, predictive, and surrogate biomarkers, a blood test, a urine test, and a genetic test. In some embodiments, performing the biomarker test comprises obtaining biological samples from the treatment set of subjects and the reference set of subjects, and assaying the biological samples to determine the treatment outcomes.

In some embodiments, the method further comprises comparing treatment outcomes between the first subject and the second subject for each of a plurality of clinical interventions, and prioritizing or ranking the plurality of clinical interventions for the subject. In some embodiments, the treatment outcomes comprise a plurality of endpoints. In some embodiments, the plurality of endpoints is prioritized or ranked. In some embodiments, the set of pairwise comparisons comprises all possible pairwise combinations of a subject selected from the treatment set and a subject selected from the reference set.

In some embodiments, the prioritization function in (c) is selected by the subject. In some embodiments, the prioritization function in (c) is selected based on at least one of efficacies, adverse effects, and/or thresholds of clinical relevance of individual treatment outcomes of the plurality of treatment outcomes. In some embodiments, the prioritization function in (c) comprises at least one of an ordering, a ranking, a set of weights, and a non-transitive ordering for individual treatment outcomes of the plurality of treatment outcomes. In some embodiments, the set of weights comprises non-zero weights.

In some embodiments, the method further comprises selecting a clinical intervention from among a plurality of clinical interventions to be administered or provided to the subject, based at least in part on the net treatment benefit determined in (e). In some embodiments, the method further comprises prescribing the clinical intervention to the subject based at least in part on the net treatment benefit determined in (e). In some embodiments, the method further comprises administering or providing the clinical intervention to the subject based at least in part on the net treatment benefit determined in (e).

In some embodiments, the treatment set of subjects and the reference set of subjects each comprise at least 10 subjects, at least 50 subjects, at least 100 subjects, etc.

In some embodiments, the clinical intervention is part of a clinical trial.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates an example of a method for identifying a net treatment benefit of a clinical intervention for a subject.

FIG. 2 illustrates a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an”, and “the” include plural references unless the context clearly dictates otherwise. For example, the term “an intervention” includes a plurality of interventions.

As used herein, the term “subject,” generally refers to an entity or a medium that has testable or detectable genetic information. A subject may be a person, individual, or patient. A subject may be a vertebrate, such as, for example, a mammal. Non-limiting examples of mammals include humans, simians, farm animals, sport animals, rodents, and pets. The subject may be displaying a symptom(s) indicative of a health or physiological state or condition of the subject. As an alternative, the subject may be asymptomatic with respect to such health or physiological state or condition. The subject may be an adult or a child.

The present disclosure provides improved methods and systems for carrying out statistical analysis on clinical data, which may allow the consideration of a plurality of outcome interests. Inputs from a user, such as a clinician, a medical professional, or a patient may be incorporated into the statistical analysis so that statistical inferences over the underlying clinical data may be made with granularity. For example, statistical inferences may be made on a case by case scenario based on a user's preferences, needs, wants, or a combination thereof.

In an aspect, the present disclosure provides a computer-implemented method for identifying a net treatment benefit of a clinical intervention for a subject, comprising: obtaining a dataset for a treatment set of subjects and a reference set of subjects, wherein the treatment set of subjects receives the clinical intervention, and wherein the reference set of subjects does not receive the clinical intervention; obtaining a plurality of treatment outcomes for the treatment set of subjects and the reference set of subjects; receiving user input of a prioritization function of the plurality of treatment outcomes; performing a set of pairwise comparisons between a first subject selected from the treatment set of subjects and a second subject selected from the reference set of subjects, at least in part by performing a prioritized comparison of the plurality of treatment outcomes between the first subject and the second subject based at least in part on the prioritization function; and determining the net treatment benefit of the clinical intervention for the subject, based at least in part on the set of pairwise comparisons. In some embodiments, the clinical intervention comprises a medication (e.g., topical, intracavitary, oral or systemic drug or biological treatment), cell-based or gene therapy, immunotherapy, a medical device (e.g., laser or photodynamic therapy, radiofrequency or thermal ablation, or cryotherapy), a surgical intervention (e.g., invasive, minimally invasive, laparoscopic, or others), radiotherapy, radioisotopic:/nuclear therapy, physical therapy, occupational therapy, phonoaudiological therapy, a rehabilitation intervention, a digital health intervention, a behavioral intervention, a psychological intervention, or any combination thereof. In some embodiments, the clinical intervention comprises a drug treatment. In some embodiments, the drug treatment comprises an approved (e.g., approved) drug treatment. In some embodiments, drug treatment comprises an experimental drug treatment. In some embodiments, the clinical intervention comprises an intervention that may be compared between a case group and a control group. In some embodiments, the clinical intervention comprises an intervention that may be compared between one case group and another case group. In some embodiments, the clinical intervention is part of a clinical trial. In some embodiments, the treatment set of subjects and/or the reference set of subjects correspond to one or more randomized clinical trials.

In some embodiments, the net treatment benefit comprises a therapeutic benefit, selfreported satisfaction of a subject, reducing risk of a treatment, an increase in survivability, an increase in self-perceived well-being, an increase in well-being observed by a medical professional, an increase in self-perceived happiness, an increase in happiness observed by a medical professional, an increase in self-perceived mental health, an increase in mental health observed by a medical professional, an increase in physical activity, an increase in mental activity, an increase in sleep quality, an increase in metabolism, an increase in brain activity, an increase in hearing ability, an increase in sense of smell, an increase in visual acuity, an increase in the ability to taste, an increased cell regeneration, an increase in immune system strength, an increase in blood flow, an increased appetite, a reduction in pain, a reduction in physical pain, a reduction in the severity of mental issues, a reduction in the amount of negative thoughts, a reduction in the amount of suicidal thoughts, a reduction in anxiety, a reduction in cancer cells, a reduction in infection, a reduction in frequency of psychotic episodes, a reduction in frequency of seizures, an improvement in the strength of muscles, an improvement in the strength of involuntary muscles, an improvement in the strength of the diaphragm, an improvement in the strength of the heart, an improvement in the strength of the digestive tract, any other change in symptoms, clinical outcomes or biomarker values, or any combination thereof.

In some embodiments, the clinical intervention comprises a medication (e.g., topical, intracavitary, oral or systemic drug or biological treatment), cell-based or gene therapy, immunotherapy, a medical device (e.g., laser or photodynamic therapy, radiofrequency or thermal ablation, or cryotherapy), a surgical intervention (e.g., invasive, minimally invasive, laparoscopic, or others), radiotherapy, radioisotopic/nuclear therapy, physical therapy, occupational therapy, phonoaudiological therapy, a rehabilitation intervention, a digital health intervention, a behavioral intervention, a psychological intervention, or any combination thereof. In some embodiments, the clinical intervention comprises an approved treatment. In some embodiments, the clinical intervention comprises an experimental treatment. In some embodiments, the clinical intervention comprises an approved drug treatment. In some embodiments, the clinical intervention comprises an experimental drug treatment. In some embodiments, the clinical intervention comprises an intervention that may be compared between a case group and a control group. In some embodiments, the clinical intervention comprises an intervention that may be compared between one case group and another case group. In some embodiments, the clinical intervention is part of a clinical trial. In some embodiments, the treatment set of subjects and/or the reference set of subjects correspond to one or more randomized clinical trials.

Various forms of data may be used to measure treatment outcomes. In some embodiments, treatment outcomes are measured by discrete variables or continuous variables. In some embodiments, treatment outcomes are measured by categorical variables, true or false variables, color variables, or any combination thereof. Any number of variables may be used to measure a treatment outcome. In some embodiments, a treatment outcome is measured by 1 variable, 2 variables, 3 variables, 4 variables, 5 variables, 6 variables, 7 variables, 8 variables, 9 variables, 10 variables, or more than 10 variables.

The plurality of treatment outcomes may include various kinds of treatment outcomes. In some embodiments, the treatment outcomes comprise an event-free survival time, a progression-free survival time, an overall survival time, another time to an event, efficacy, safety, quality of life, a score (functional score, performance score, toxicity grade, behavioral score, a composite score, an index score, or a combination thereof), cost-related information, or levels or a change in levels of a biomarker or any combination thereof.

In some embodiments, an event-free survival time may refer to the duration of time between an end of a treatment and future occurrence of a medical event related to the treatment or the condition the treatment is designed to treat. In some embodiments, an event-free survival time may refer to the duration of time between an end of a treatment and death related to the treatment or the condition the treatment is designed to treat. In some embodiments, an event-free survival time may refer to the duration of time between an end of a treatment and death related to natural causes. In some embodiments, progression-free survival time may refer to the duration of time between an end of a treatment and a progression in the condition that the treatment is designed to treat. In some embodiments, progression-free survival time may refer to the duration of time between an end of a treatment and a progression in the condition that the treatment is designed to treat. In some embodiments, a progression-free survival time may refer to the duration of time between an end of a treatment and death related to natural causes.

In some embodiments, efficacy may refer to the efficacy of a treatment in providing a therapeutic benefit. In some embodiments, a therapeutic benefit comprises an increase in self perceived well-being, an increase in well-being observed by a medical professional, an increase in self-perceived happiness, an increase in happiness observed by a medical professional, an increase in self-perceived mental health, an increase in mental health observed by a medical professional, an increase in physical activity, an increase in mental activity, an increase in sleep quality, an increase in metabolism, an increase in brain activity, an increase in hearing ability, an increase in sense of smell, an increase in visual acuity, an increase in the ability to taste, an increase cell regeneration, an increase in immune system strength, an increase in blood flow, an increased appetite, a reduction in pain, a reduction in physical pain, a reduction in the severity of mental issues, a reduction in the amount of negative thoughts, a reduction in the amount of suicidal thoughts, a reduction in anxiety, a reduction in cancer cells, a reduction in infection, a reduction in frequency of psychotic episodes, a reduction in frequency of seizures, an improvement in the strength of muscles, an improvement in the strength of involuntary muscles, an improvement in the strength of the diaphragm, an improvement in the strength of the heart, an improvement in the strength of the digestive tract, or any combination thereof.

In some embodiments, safety may refer to the chances of an undesirable medical condition manifesting in a patient during or after a treatment. In some embodiments, safety may refer to the severity of an undesirable medical condition manifesting in a patient during or after a treatment. In some cases, an undesirable medical condition comprises a drug side effect, an undesirable surgical outcome, development of chronic conditions, development of an infection, development of a mental conditions, development of an addiction, loss of hair, loss of a limb, loss of tissue, loss of an organ, loss of blood, pain, death, decrease in a biomarker, or any combination thereof.

In some embodiments, quality of life comprises the amount of physical activity by a subject, the amount of mental activity by a subject, self-reported levels of happiness from a subject, self-reported levels of well-being from a subject, amount of sleeping hours by a subject, amount of waking hours by a subject, amount of time a subject may spend with friends and family, amount of pain felt by a subject, or any combination thereof.

In some embodiments, a biomarker comprises a chemical, genomic, epigenomic, gene expression, protein, or a metabolic biomarker. In some embodiments, a biomarker comprises an amino acid, histidine, isoleucine, leucine, lysine, methionine, cysteine, phenylalanine, tyrosine, threonine, tryptophan, valine, arginine, glutamine, alanine, aspartic acid, aspartate, asparagine, glutamic acid, glutamate, proline, serine, glycine, alanine, a peptide, a protein, a nucleotide, cytosine, adenine, guanine, thymine, uracil, a nucleic acid, single-stranded DNA, double-stranded DNA, single-stranded RNA, double-stranded RNA, mRNA, tRNA, rRNA, cRNA, ncRNA, lncRNA, snoRNA, snRNA, piRNA, siRNA, miRNA, a phosphorylated protein and nucleic acid complex, a methylated protein and nucleic acid complex, a monosaccharide, glucose, fructose, galactose, a polysaccharide, glycogen, a carbohydrate, a lipid, a phospholipid, a fatty acid, a cholesterol, 02, CO2, a hormone, adrenaline, melatonin, noradrenaline, triiodothyronine, thyroxine, prostaglandin, leukotriene, prostacyclin, thromboxane, amylin, adrenocorticotropic hormone, angiotensinogen, vasopressin, atriopeptin, brain natriuretic, calcitonin, cholecystokinin, corticotropin-releasing hormone, cortistatin, enkephalin, endothelin, erythropoietin, galanin, gastrin, ghrelin, glucagon, gonadotropin-releasing hormone, growth horming, hepcidin, gonadotropin, lactogen, inhibin, insulin, somatomedin, leptin, lipotropin, motilin, orexin, osteocalcin, parathyroid hormone, prolactin, relaxin, renin, secretinin, somatostatin, thrombopoietin, thyrotropin, guanylin, uroguanylin, serotonin, dopamine, oxytocin, endorphin, a steroid, testosterone, estrogen, dehydroepiandrosterone, androstenedione, dihydrotestosterone, aldosterone, estradiol, estrone, estriol, progesterone, calcitriol, calcidiol, sodium, potassium, magnesium, manganese, selenium, copper, chromium, fluoride, chloride, lithium, beryllium, calcium, bromide, iodide, acetone, methanol, ethanol, or any combination thereof.

In some embodiments, a clinical test result comprises amniocentesis, blood analysis, blood count, blood typing, bone marrow aspiration, cephalin-cholesterol flocculation, enzyme analysis, epinephrine tolerance test, glucose tolerance test, hematocrit, immunologic blood test, inulin clearance, serological test, thymol turbidity, gastric fluid analysis, kidney function test, liver function test, lumbar puncture, malabsorption test, Pap smear, phenolsulfonphthalein test, pregnancy test, prenatal testing, protein-bound iodine test, syphilis test, thoracentesis, thyroid function test, toxicology test, urinalysis/uroscopy, diagnostic imaging, angiocardiography, angiography, cerebral angiography, brain scanning, echoencephalography, magnetoencephalography, pneumoencephalography, cholecystography, echocardiography, endoscopic retrograde cholangiopancreatoscopy, lung ventilation/perfusion scan, magnetic resonance imaging, cardiac magnetic resonance imaging, functional magnetic resonance imaging, magnetic resonance spectroscopy, mammography, myelography, prenatal testing, tomography, computed tomography, positron emission tomography, single photon emission computed tomography, ultrasound, urography, genetic testing, complementation test, fluorescence in situ hybridization, preimplantation genetic diagnosis, measurement, ballistocardiography, electrocardiography, electroencephalography, electromyography, lumbar puncture, magnetic resonance spectroscopy, phonocardiography, pulmonary function test, semen analysis, physical and visual examination, auscultation, autopsy, biopsy, bronchoscopy, cardiac catheterization, colposcopy, Dick test, endoscopy, esophagogastroduodenoscopy, gynecological examination, laparoscopy, mediastinoscopy, nasopharyngolaryngoscopy, palpation, percussion, Rubin's test, semen analysis, skin test, patch test, Schick test, tuberculin test, toxicological examination, uroscopy, or any combination thereof.

In some embodiments, the method comprises determining a difference in the treatment outcomes between the first subject and the second subject. In some embodiments, a subject comprises a patient being treated (e.g., a past patient that was previously treated, a present patient that is being presently treated, a future patient that may receive treatment in the future, or combination thereof). In some cases, a subject may have one or more medical conditions. In some embodiments, the treatment set of subjects and the reference set of subjects comprise subjects having a disease or disorder. In some embodiments, the disease, the disorder, or the medical condition comprises cancer, tumors, acute lymphoblastic leukemia, acute myeloid leukemia, adrenocortical carcinoma, AIDS-related cancer, kaposi sarcoma, AIDS -related lymphoma, primary central nervous system lymphoma, anal cancer, appendix cancer, astrocytomas, atypical teratoid, rhabdoid tumor, basal cell carcinoma, bile duct cancer, bladder cancer, bone cancer, Ewing sarcoma, osteosarcoma, malignant fibrous histiocytoma, brain tumors, breast cancer, bronchial tumors, Burkitt lymphoma, carcinoid tumor, cardiac tumors, atypical teratoid tumor, medulloblastoma, central nervous system embryonal tumors, germ cell tumor, cervical cancer, cholangiocarcinoma, bile duct cancer, chordoma, chronic lymphocytic leukemia, chronic myelogenous leukemia, chronic myeloproliferative neoplasms, colorectal cancer, craniopharyngioma, cutaneous T-cell lymphoma, ductal carcinoma, medulloblastoma, endometrial cancer, uterine cancer, ependymoma, esophageal cancer, esthesioneuroblastoma, Ewing sarcoma, extracranial germ cell tumor, eye cancer, intraocular melanoma, retinoblastoma, fallopian tube cancer, gallbladder cancer, gastric cancer, gastrointestinal carcinoid tumor, gastrointestinal stromal tumors, germ cell tumors, childhood extracranial germ cell tumors, extragonadal germ cell tumors, ovarian germ cell tumors, testicular cancer, gestational trophoblastic disease, hairy cell leukemia, head and neck cancer, heart tumors, hepatocellular cancer, histiocytosis, Hodgkin lymphoma, hypopharyngeal cancer, islet cell tumors, pancreatic neuroendocrine tumors, Kaposi sarcoma, kidney cancer, Langerhans cell histiocytosis, laryngeal cancer, leukemia, lip and oral cavity cancer, lung cancer, non-small cell lung cancer, small cell lung cancer, pleuropulmonary blastoma, tracheobronchial tumor, male breast cancer, melanoma, Merkel cell carcinoma, mesothelioma, metastatic cancer, metastatic squamous neck cancer, midline tract carcinoma, mouth cancer, multiple endocrine neoplasia syndromes, multiple myeloma/plasma cell neoplasms, mycosis fungoides, myelodysplastic syndromes, myelodysplastic/myeloproliferative neoplasms, myelogenous leukemia, myeloid leukemia, myeloproliferative neoplasms, nasal cavity and paranasal sinus cancer, nasopharyngeal cancer, neuroblastoma, non-Hodgkin lymphoma, non small cell lung cancer, oral cancer, oropharyngeal cancer, osteosarcoma, undifferentiated pleomorphic sarcoma, ovarian cancer, pancreatic cancer, pancreatic neuroendocrine tumors, islet cell tumors, papillomatosis, paraganglioma, paranasal sinus and nasal cavity cancer, parathyroid cancer, penile cancer, pharyngeal cancer, pheochromocytoma, pituitary tumor, plasma cell neoplasm/multiple myeloma, pleuropulmonary blastoma, pregnancy cancer, primary peritoneal cancer, prostate cancer, rectal cancer, recurrent cancer, retinoblastoma, rhabdomyosarcoma, salivary gland cancer, sarcoma, childhood rhabdomyosarcoma, childhood vascular tumors, Ewing sarcoma, Kaposi sarcoma, osteosarcoma, soft tissue sarcoma, uterine sarcoma, Sezary syndrome, skin cancer, small intestine cancer, soft tissue sarcoma, squamous cell carcinoma of the skin, squamous neck cancer with occult primary, stomach cancer, t-cell lymphoma, testicular cancer, throat cancer, nasopharyngeal cancer, oropharyngeal cancer, hypopharyngeal cancer, thymoma and thymic carcinoma, thyroid cancer, tracheobronchial tumors, transitional cell cancer of the renal pelvis and ureter, urethral cancer, endometrial uterine cancer, uterine sarcoma, vaginal cancer, vascular tumors, vulvar cancer, Wilms tumor, a genetic disorder, 1p36 deletion syndrome, 1q21.1 deletion syndrome, 2q37 deletion syndrome, 5q deletion syndrome, 5,10-methenyltetrahydrofolate synthetase deficiency, 17q12 microdeletion syndrome, 17q12 microduplication syndrome, 18p deletion syndrome, 21-hydroxylase deficiency, Alpha 1-antitrypsin deficiency, AAA syndrome (achalasia-Addisonianism-alacrima syndrome), Aarskog-Scott syndrome, ABCD syndrome, Aceruloplasminemia, Acheiropodia, Achondrogenesis type II, achondroplasia, Acute intermittent porphyria, Adenylosuccinate lyase deficiency, Adrenoleukodystrophy, Alagille syndrome, ADULT syndrome, Aicardi-Goutieres syndrome, Albinism, Alexander disease, Alfi's syndrome, alkaptonuria, Alport syndrome, Alternating hemiplegia of childhood, Amyotrophic lateral sclerosis - Frontotemporal dementia, Alstrom syndrome, Alzheimer's disease, Amelogenesis imperfecta, Aminolevulinic acid dehydratase deficiency porphyria, Androgen insensitivity syndrome, Angelman syndrome, Apert syndrome, Arthrogryposis-renal dysfunction-cholestasis syndrome, Ataxia telangiectasia, Axenfeld syndrome, Beare-Stevenson cutis gyrata syndrome, Beckwith-Wiedemann syndrome, Benjamin syndrome, biotinidase deficiency, Bjornstad syndrome, Bloom syndrome, Birt-Hogg-Dube syndrome, Brody myopathy, Brunner syndrome, CADASIL syndrome, Cat eye syndrome, CRASIL syndrome, Chronic granulomatous disorder, Campomelic dysplasia, Canavan disease, Carpenter Syndrome, CDKL5 deficiency disorder, Cerebral dysgenesis neuropathy-ichthyosis-keratoderma syndrome (CEDNIK), Cystic fibrosis, Charcot-Marie-Tooth disease, CHARGE syndrome, Chediak-Higashi syndrome, Chondrodysplasia, Grebe type, Cleidocranial dysostosis, Cockayne syndrome, Coffin-Lowry syndrome, Cohen syndrome, collagenopathy, types II and XI, Congenital insensitivity to pain with anhidrosis (CIPA), Congenital Muscular Dystrophy, Cornelia de Lange syndrome (CDLS), Cowden syndrome, CPO deficiency (coproporphyria), Cranio-lenticulo-sutural dysplasia, Cri du chat, Crohn's disease, Crouzon syndrome, Crouzonodermoskeletal syndrome (Crouzon syndrome with acanthosis nigricans), Currarino syndrome, Darier's disease, Dent's disease (Genetic hypercalciuria), Denys-Drash syndrome, De Grouchy syndrome, Down Syndrome, DiGeorge syndrome, Distal hereditary motor neuropathies, multiple types, Distal muscular dystrophy, Duchenne muscular dystrophy, Dravet syndrome, Edwards Syndrome, Ehlers-Danlos syndrome, Emanuel syndrome, Emery-Dreifuss syndrome, Epidermolysis bullosa, Erythropoietic protoporphyria, Fanconi anemia (FA), Fabry disease, Factor V Leiden thrombophilia, Fatal familial insomnia, Familial adenomatous polyposis, Familial dysautonomia, Familial Creutzfeld-Jakob Disease, Feingold syndrome, FG syndrome, Fragile X syndrome, Friedreich's ataxia, G6PD deficiency, Galactosemia, Gaucher disease, Gerstmann-Straussler-Scheinker syndrome, Gillespie syndrome, Glutaric aciduria, type 1 and type 2, GRACILE syndrome, Griscelli syndrome, Hailey-Hailey disease, Harlequin type ichthyosis, Hemochromatosis type 1, Hemochromatosis type 2A, Hemochromatosis type 2B, Haemochromatosis type 3, Hemochromatosis type 4, Hemochromatosis type 5, Hemophilia, Hepatoerythropoietic porphyria, Hereditary coproporphyria, Hereditary hemorrhagic telangiectasia (Osler-Weber-Rendu syndrome), Hereditary inclusion body myopathy, Hereditary multiple exostoses, Hereditary spastic paraplegia (infantile-onset ascending hereditary spastic paralysis), Hermansky-Pudlak syndrome, Hereditary neuropathy with liability to pressure palsies (HNPP), Heterotaxy, Homocystinuria, Huntington's disease, Hunter syndrome, Hurler syndrome, Hutchinson-Gilford progeria syndrome, Hyperlysinemia, Hyperoxaluria, primary, Hyperphenylalaninemia, Hypoalphalipoproteinemia (Tangier disease), Hypochondrogenesis, Hypochondroplasia, Immunodeficiency-centromeric instability-facial anomalies syndrome (ICF syndrome), Incontinentia pigmenti, Ischiopatellar dysplasia, Isodicentric 15, Jackson-Weiss syndrome, Jacobsen syndrome, Joubert syndrome, Juvenile primary lateral sclerosis (JPLS), Keloid disorder, Kleefstra syndrome, Kniest dysplasia, Kosaki overgrowth syndrome, Krabbe disease, Kufor-Rakeb syndrome, LCAT deficiency, Lesch-Nyhan syndrome, Li-Fraumeni syndrome, Limb Girdle Muscular Dystrophy, Lynch syndrome, lipoprotein lipase deficiency, Malignant hyperthermia, Maple syrup urine disease, Marfan syndrome, Maroteaux-Lamy syndrome, McCune Albright syndrome, McLeod syndrome, MEDNIK syndrome, Mediterranean fever, familial, Menkes disease, Methemoglobinemia, Methylmalonic acidemia, Micro syndrome, Microcephaly, Miller Dieker syndrome, Morquio syndrome, Mowat-Wilson syndrome, Muenke syndrome, Multiple endocrine neoplasia type 1 (Wermer's syndrome), Multiple endocrine neoplasia type 2, Muscular dystrophy, Muscular dystrophy, Duchenne and Becker type, Myostatin-related muscle hypertrophy, myotonic dystrophy, Natowicz syndrome, Neurofibromatosis type I, Neurofibromatosis type II, Niemann-Pick disease, Nonketotic hyperglycinemia, Nonsyndromic deafness, Noonan syndrome, Norman-Roberts syndrome, Ogden syndrome, Omenn syndrome, Osteogenesis imperfecta, Pantothenate kinase-associated neurodegeneration, Patau syndrome (Trisomy 13), PCC deficiency (propionic acidemia), Porphyria cutanea tarda (PCT), Pendred syndrome, Peutz-Jeghers syndrome, Pfeiffer syndrome, Phelan-McDermid syndrome, Phenylketonuria, Pipecolic acidemia, Pitt-Hopkins syndrome, Polycystic kidney disease, Polycystic ovary syndrome (PCOS), Porphyria, Prader-Willi syndrome, Primary ciliary dyskinesia (PCD), Primary pulmonary hypertension, Protein C deficiency, Protein S deficiency, Proximal 18q deletion syndrome, Pseudo-Gaucher disease, Pseudoxanthoma elasticum, Retinitis pigmentosa, Rett syndrome, Roberts syndrome, Rubinstein Taybi syndrome (RSTS), Sandhoff disease, Sanfilippo syndrome, Schwartz-Jampel syndrome, Sjogren-Larsson syndrome, Spondyloepiphyseal dysplasia congenita (SED), Shprintzen-Goldberg syndrome, Sickle cell anemia, Siderius X-linked mental retardation syndrome, Sideroblastic anemia, Sly syndrome, Smith-Lemli-Opitz syndrome, Smith-Magenis syndrome, Snyder-Robinson syndrome, Spinal muscular atrophy, Spinocerebellar ataxia (types 1-29), SSB syndrome (SADDAN), Stargardt disease (macular degeneration), Stickler syndrome (multiple forms), Strudwick syndrome (spondyloepimetaphyseal dysplasia, Strudwick type), Tay-Sachs disease, Tetrahydrobiopterin deficiency, Thanatophoric dysplasia, Treacher Collins syndrome, Tuberous sclerosis complex (TSC), Turner syndrome, Usher syndrome, Variegate porphyria, von HippelLindau disease, von Willebrand disease, Waardenburg syndrome, Warkany syndrome 2, Weissenbacher-Zweymuller syndrome, Williams syndrome, Wilson disease, Woodhouse-Sakati syndrome, Wolf-Hirschhorn syndrome, Xeroderma pigmentosum, X-linked intellectual disability and macroorchidism (fragile X syndrome), X-linked spinal-bulbar muscle atrophy (spinal and bulbar muscular atrophy), Xp11.2 duplication syndrome, X-linked severe combined immunodeficiency (X SCID), X-linked sideroblastic anemia (XLSA), 47,XXX (triple X syndrome), XXXX syndrome (48, XXXX), XXXXX syndrome (49,XXXXX), XXXXY syndrome (49,XXXXY), XYY syndrome (47,XYY), XXYY syndrome (48,XXYY), XYYY syndrome (48,XYYY), XXXY syndrome (48,XXXY), XYYYY syndrome (49,XYYYY), Zellweger syndrome, liveborn, septicemia, heart failture, osteoarthritis, pneumonia, diabetes, acute myocardial infraction, cardiac dysrhythmia, chronic obstructive pulmonary disease, bronchiectasis, congestive heart failure, mood disorder, implant complications, graft complications, coronary atherosclerosis, cardiovascular disease, heart disease, or any combination thereof. In some embodiments, the disease, the disorder, or the medical condition is an adult disease, disorder, or medical condition. In some embodiments, the disease, the disorder, or the medical condition is a child disease, disorder, or medical condition.

In some embodiments, the method comprises comparing the difference in the treatment outcomes between the first subject and the second subject to a clinical threshold. In some embodiments, the method comprises comparing each of a plurality of treatment outcomes between the first subject and the second subject. In some embodiments, the method comprises characterizing a pairwise comparison as a win, a loss, a tie, or an indeterminate comparison based at least in part on the difference in the treatment outcomes between the first subject and the second subject. In some embodiments, the pairwise comparison is characterized as a win, a loss, or a tie based at least in part on the difference in the treatment outcomes being a positive difference greater than a threshold, a negative difference greater than a threshold, or a difference less than a threshold, respectively. In some embodiments, the method comprises determining a likelihood that the first subject has a better treatment outcome than the second subject, based at least in part on the set of pairwise comparisons. In some embodiments, the likelihood comprises a relative likelihood (e.g., a likelihood ratio or odds ratio, such as win ratio or win odds), an absolute likelihood, or a net likelihood (e.g., a difference between two likelihoods, such as net benefit). In some embodiments, the likelihood comprises a probability or a net probability (e.g., a difference between two probabilities).

In some embodiments, the clinical threshold comprises a minimal threshold for a positive outcome. In some embodiments, the clinical threshold comprises a minimal threshold for a clinically worthwhile effect. In some embodiments, the clinical threshold comprises an outcome of another treatment.

In some embodiments, the method comprises comparing the treatment outcomes between the first subject and the second subject at least in part by comparing a net benefit minus a net harm between the first subject and the second subject. In some embodiments, the net benefit comprises event-free survival time, progression-free survival time, overall survival time, efficacy, safety, quality of life, or any combination thereof.

In some embodiments, the net harm comprises an adverse event grade comprising a side effect of the clinical intervention, a toxicity of the clinical intervention, or any combination thereof. In some embodiments, a side effect comprises constipation, skin rash, dermatitis, diarrhea, dizziness, drowsiness, dry mouth, headache, insomnia, nausea, suicidal thoughts, abnormal heart rhythms, internal bleeding, fever, loss in appetite, increase in psychotic episodes, dissociative disorder, fatigue, swelling, allergic reactions, decrease in sex drive, infertility, menopause, alopecia, reduction in memory, reduction in attention span, hearing impairment, low blood platelet count, low red blood cell count, low white blood cell count, mucositis, moodiness, dry skin, erectile dysfunction, nerve damage, infection, or any combination thereof. In some cases toxicity comprises chemical toxicity, biological toxicity, physical toxicity, radiation toxicity, behavioral toxicity, or any combination thereof.

In some embodiments, the treatment outcomes are obtained by performing a biomarker test on the treatment set of subjects and the reference set of subjects.

In some embodiments, the biomarker test comprises test or a measurement for a radiological test, a blood test, a urine test, a genetic test, an epigenomic test, gene expression test, protein expression test, or a metabolic biomarker test, a biopsy, a spinal tap, a tear test, a saliva test, or any combination thereof. In some embodiments, a biomarker test comprises testing for an amino acid, histidine, isoleucine, leucine, lysine, methionine, cysteine, phenylalanine, tyrosine, threonine, tryptophan, valine, arginine, glutamine, alanine, aspartic acid, aspartate, asparagine, glutamic acid, glutamate, proline, serine, glycine, alanine, a peptide, a protein, a nucleotide, cytosine, adenine, guanine, thymine, uracil, a nucleic acid, single-stranded DNA, double-stranded DNA, single stranded RNA, double-stranded RNA, mRNA, tRNA, rRNA, cRNA, ncRNA, lncRNA, snoRNA, snRNA, piRNA, siRNA, miRNA, a phosphorylated protein and nucleic acid complex, a methylated protein and nucleic acid complex, a monosaccharide, glucose, fructose, galactose, a polysaccharide, glycogen, a carbohydrate, a lipid, a phospholipid, a fatty acid, a cholesterol, 02, CO2, a hormone, adrenaline, melatonin, noradrenaline, triiodothyronine, thyroxine, prostaglandin, leukotriene, prostacyclin, thromboxane, amylin, adrenocorticotropic hormone, angiotensinogen, vasopressin, atriopeptin, brain natriuretic, calcitonin, cholecystokinin, corticotropin-releasing hormone, cortistatin, enkephalin, endothelin, erythropoietin, galanin, gastrin, ghrelin, glucagon, gonadotropin releasing hormone, growth horming, hepcidin, gonadotropin, lactogen, inhibin, insulin, somatomedin, leptin, lipotropin, motilin, orexin, osteocalcin, parathyroid hormone, prolactin, relaxin, renin, secretinin, somatostatin, thrombopoietin, thyrotropin, guanylin, uroguanylin, serotonin, dopamine, oxytocin, endorphin, a steroid, testosterone, estrogen, dehydroepiandrosterone, androstenedione, dihydrotestosterone, aldosterone, estradiol, estrone, estriol, progesterone, calcitriol, calcidiol, sodium, potassium, magnesium, manganese, selenium, copper, chromium, fluoride, chloride, lithium, beryllium, calcium, bromide, iodide, acetone, methanol, ethanol, or any combination thereof.

In some embodiments, performing the biomarker test comprises obtaining biological samples from the treatment set of subjects and the reference set of subjects, and assaying the biological samples to determine the treatment outcomes.

In some embodiments, the method comprises comparing treatment outcomes between the first subject and the second subject for each of a plurality of clinical interventions, and prioritizing or ranking the plurality of clinical interventions for the subject.

In some embodiments, prioritizing or ranking comprises assigning a number to each clinical intervention, wherein the number corresponds to a priority value or a rank value of each clinical intervention. In some embodiments, the number may be an integer. In some embodiments, no two clinical interventions are assigned the same number. In some embodiments, at least two clinical interventions may be assigned the same number. In some embodiments, prioritizing or ranking comprises assigning a plurality of numbers to each clinical intervention. In some embodiments, the prioritization function is selected by a subject. In some embodiments, the prioritization function is selected by a clinician. In some embodiments, the prioritization function is selected by a health care professional. In some embodiments, the prioritization function is selected by a patient. In some embodiments, the prioritization function is selected by a patient representative. In some embodiments, the prioritization function is selected by a member of the general public. In some embodiments, the prioritization function is selected based on at least one of efficacies, adverse effects, and/or thresholds of clinical relevance of individual treatment outcomes of the plurality of treatment outcomes. In some embodiments, the prioritization function comprises at least one of an ordering, a ranking, a set of weights, and a non-transitive ordering for individual treatment outcomes of the plurality of treatment outcomes.

In some embodiments, the set of weights comprises non-zero weights. In some embodiments, the set of weights comprises zero weights. In some embodiments, weights are expressed as a value between 0 and 1. In some embodiments, weights are expressed in percentages (e.g., with the sum of weights adding to 1).

In some embodiments, a non-transitive ordering may refer to an ordering with binary relations that are not transitive relations. In some embodiments, a non-transitive ordering may be synthesized from the plurality of treatment outcomes. In some embodiments, a non-transitive ordering may be synthesized from the plurality of treatment outcomes and a prioritization order or ranking of the plurality of treatment outcomes.

In some embodiments, the treatment outcomes comprise a plurality of endpoints. In some embodiments, the plurality of endpoints each comprise a measurement of a treatment progress or a treatment outcome. In some embodiments, the plurality of endpoints each comprise a projected treatment progress or a projected treatment outcome. In some embodiments, the plurality of endpoints each comprise one or more assays of a subject, a test of a subject, a medical examination of a subject, and any reports thereof. In some embodiments, each endpoint in the plurality of endpoints is created when a subject visits a hospital and receives a diagnostic, a consultation, a treatment, a therapy, or any combination thereof.

In some embodiments, the plurality of endpoints is prioritized or ranked. In some embodiments, the plurality of endpoints is prioritized or ranked by a user.

In some embodiments, the set of pairwise comparisons comprises all possible pairwise combinations of a subject selected from the treatment set and a subject selected from the reference set. In some embodiments, the set of pairwise comparisons comprises all possible pairwise combinations of a subject selected from the treatment set and a subject selected from the treatment set. In some embodiments, the set of pairwise comparisons comprises all possible pairwise combinations of a subject selected from the treatment set plus the reference set and a subject selected from the treatment set plus the reference set.

In some embodiments, the method further comprises selecting a clinical intervention from among a plurality of clinical interventions to be administered or provided to the subject, based at least in part on the net treatment benefit. In some embodiments, the method further comprises prescribing the clinical intervention to the subject based at least in part on the net treatment benefit. In some embodiments, the method further comprises administering or providing the clinical intervention to the subject based at least in part on the net treatment benefit.

In some embodiments, the treatment set of subjects comprise at least 1 subject, at least 2 subjects, at least 3 subjects, at least 4 subjects, at least 5 subjects, at least 6 subjects, at least 7 subjects, at least 8 subjects, at least 9 subjects, at least 10 subjects, at least 50 subjects, at least 100 subjects, at least 150 subjects, at least 200 subjects, at least 250 subjects, at least 300 subjects, at least 350 subjects, at least 400 subjects, at least 450 subjects, at least 500 subjects, at least 550 subjects, at least 600 subjects, at least 650 subjects, at least 700 subjects, at least 750 subjects, at least 800 subjects, at least 850 subjects, at least 900 subjects, at least 950 subjects, at least 1000 subjects, at least 2000 subjects, at least 3000 subjects, at least 4000 subjects, at least 5000 subjects, at least 6000 subjects, at least 7000 subjects, at least 8000 subjects, at least 9000 subjects, at least 10000 subjects, at least 20000 subjects, at least 30000 subjects, at least 40000 subjects, at least 50000 subjects, at least 60000 subjects, at least 70000 subjects, at least 80000 subjects, at least 90000 subjects, at least 100000 subjects, at least 200000 subjects, at least 300000 subjects, at least 400000 subjects, at least 500000 subjects, at least 600000 subjects, at least 700000 subjects, at least 800000 subjects, at least 900000 subjects, at least 1000000 subjects, at least 2000000 subjects, at least 3000000 subjects, at least 4000000 subjects, at least 5000000 subjects, at least 6000000 subjects, at least 7000000 subjects, at least 8000000 subjects, at least 9000000 subjects, or at least 10000000 subjects. In some embodiments, the reference set of subjects each comprise at least 1 subject, at least 2 subjects, at least 3 subjects, at least 4 subjects, at least 5 subjects, at least 6 subjects, at least 7 subjects, at least 8 subjects, at least 9 subjects, at least 10 subjects, at least 50 subjects, at least 100 subjects, at least 150 subjects, at least 200 subjects, at least 250 subjects, at least 300 subjects, at least 350 subjects, at least 400 subjects, at least 450 subjects, at least 500 subjects, at least 550 subjects, at least 600 subjects, at least 650 subjects, at least 700 subjects, at least 750 subjects, at least 800 subjects, at least 850 subjects, at least 900 subjects, at least 950 subjects, at least 1000 subjects, at least 2000 subjects, at least 3000 subjects, at least 4000 subjects, at least 5000 subjects, at least 6000 subjects, at least 7000 subjects, at least 8000 subjects, at least 9000 subjects, at least 10000 subjects, at least 20000 subjects, at least 30000 subjects, at least 40000 subjects, at least 50000 subjects, at least 60000 subjects, at least 70000 subjects, at least 80000 subjects, at least 90000 subjects, at least 100000 subjects, at least 200000 subjects, at least 300000 subjects, at least 400000 subjects, at least 500000 subjects, at least 600000 subjects, at least 700000 subjects, at least 800000 subjects, at least 900000 subjects, at least 1000000 subjects, at least 2000000 subjects, at least 3000000 subjects, at least 4000000 subjects, at least 5000000 subjects, at least 6000000 subjects, at least 7000000 subjects, at least 8000000 subjects, at least 9000000 subjects, or at least 10000000 subjects.

In some embodiments, the treatment set of subjects and the reference set of subjects each comprise at least 1 subject, at least 2 subjects, at least 3 subjects, at least 4 subjects, at least 5 subjects, at least 6 subjects, at least 7 subjects, at least 8 subjects, at least 9 subjects, at least 10 subjects, at least 50 subjects, at least 100 subjects, at least 150 subjects, at least 200 subjects, at least 250 subjects, at least 300 subjects, at least 350 subjects, at least 400 subjects, at least 450 subjects, at least 500 subjects, at least 550 subjects, at least 600 subjects, at least 650 subjects, at least 700 subjects, at least 750 subjects, at least 800 subjects, at least 850 subjects, at least 900 subjects, at least 950 subjects, at least 1000 subjects, at least 2000 subjects, at least 3000 subjects, at least 4000 subjects, at least 5000 subjects, at least 6000 subjects, at least 7000 subjects, at least 8000 subjects, at least 9000 subjects, at least 10000 subjects, at least 20000 subjects, at least 30000 subjects, at least 40000 subjects, at least 50000 subjects, at least 60000 subjects, at least 70000 subjects, at least 80000 subjects, at least 90000 subjects, at least 100000 subjects, at least 200000 subjects, at least 300000 subjects, at least 400000 subjects, at least 500000 subjects, at least 600000 subjects, at least 700000 subjects, at least 800000 subjects, at least 900000 subjects, at least 1000000 subjects, at least 2000000 subjects, at least 3000000 subjects, at least 4000000 subjects, at least 5000000 subjects, at least 6000000 subjects, at least 7000000 subjects, at least 8000000 subjects, at least 9000000 subjects, or at least 10000000 subjects.

In some aspects, the present disclosure describes a computer system for identifying a net treatment benefit of a clinical intervention for a subject, comprising: a database that is configured to store a plurality of treatment outcomes for a treatment set of subjects and a reference set of subjects, wherein the treatment set of subjects receives the clinical intervention, and wherein the reference set of subjects does not receive the clinical intervention; and computing device comprising one or more computer processors operatively coupled to the database, wherein the one or more computer processors are individually or collectively programmed to: (i) receive user input of a prioritization function of the plurality of net treatment outcomes via a user interface on the computing device; (ii) perform a set of pairwise comparisons between a first subject selected from the treatment set of subjects and a second subject selected from the reference set of subjects, at least in part by performing a prioritized comparison of the plurality of treatment outcomes between the first subject and the second subject based at least in part on the prioritization function selected in (c); and (iii) determine the net treatment benefit of the clinical intervention for the subject, based at least in part on the set of pairwise comparisons performed in (ii); and (iv) present the clinical intervention as part of a set of clinical interventions to the user for selection, ranking, or prioritization.

In some embodiments, a computing device comprises a mobile computer, a smartphone, smartwatch, a tablet, an electronic notebook, or a laptop.

In some embodiments, the first subject is randomly selected from the treatment set of subjects, and wherein the second subject is randomly selected from the reference set of subjects.

In some embodiments, the computer system comprises determining a difference in the treatment outcomes between the first subject and the second subject. In some embodiments, the computer system comprises comparing the difference in the treatment outcomes between the first subject and the second subject to a threshold. In some embodiments, the computer system comprises comparing each of a plurality of treatment outcomes between the first subject and the second subject.

In some embodiments, the computer system further comprises selecting a clinical intervention from among a plurality of clinical interventions to be administered or provided to the subject, based at least in part on the net treatment benefit. In some embodiments, the computer system further comprises prescribing the clinical intervention to the subject based at least in part on the net treatment benefit. In some embodiments, the computer system further comprises administering or providing the clinical intervention to the subject based at least in part on the net treatment benefit.

In some embodiments, the one or more computer processors are individually or collectively programmed to further characterize a pairwise comparison as a win, a loss, or a tie based at least in part on the difference in the treatment outcomes between the first subject and the second subject. In some embodiments, a pairwise comparison is characterized as a win, a loss, or a tie based at least in part on the difference in the treatment outcomes being a positive difference greater than a threshold, a negative difference greater than a threshold, or a difference less than a threshold, respectively.

In some embodiments, the one or more computer processors are individually or collectively programmed to further determine a likelihood that the first subject has a better treatment outcome than the second subject, based at least in part on the set of pairwise comparisons. In some embodiments, the computer system comprises comparing the treatment outcomes between the first subject and the second subject at least in part by comparing a net benefit minus a net harm between the first subject and the second subject.

In some embodiments, the one or more computer processors are individually or collectively programmed to further compare treatment outcomes between the first subject and the second subject for each of a plurality of clinical interventions, and prioritizing or ranking the plurality of clinical interventions for the subject.

In some embodiments, the treatment outcomes may be obtained by performing a diagnostic or radiological test on the treatment set of subjects and the reference set of subjects. In some embodiments, a diagnostic test comprises a test or a measurement for a radiological test, a blood test, a urine test, a genetic test, an epigenomic test, gene expression test, protein expression test, or a metabolic biomarker test, a biopsy, a spinal tap, a tear test, a saliva test, or any combination thereof. In some embodiments, a biomarker test comprises testing for an amino acid, histidine, isoleucine, leucine, lysine, methionine, cysteine, phenylalanine, tyrosine, threonine, tryptophan, valine, arginine, glutamine, alanine, aspartic acid, aspartate, asparagine, glutamic acid, glutamate, proline, serine, glycine, alanine, a peptide, a protein, a nucleotide, cytosine, adenine, guanine, thymine, uracil, a nucleic acid, single-stranded DNA, double-stranded DNA, single-stranded RNA, double stranded RNA, mRNA, tRNA, rRNA, cRNA, ncRNA, lncRNA, snoRNA, snRNA, piRNA, siRNA, miRNA, a phosphorylated protein and nucleic acid complex, a methylated protein and nucleic acid complex, a monosaccharide, glucose, fructose, galactose, a polysaccharide, glycogen, a carbohydrate, a lipid, a phospholipid, a fatty acid, a cholesterol, 02, CO2, a hormone, adrenaline, melatonin, noradrenaline, triiodothyronine, thyroxine, prostaglandin, leukotriene, prostacyclin, thromboxane, amylin, adrenocorticotropic hormone, angiotensinogen, vasopressin, atriopeptin, brain natriuretic, calcitonin, cholecystokinin, corticotropin-releasing hormone, cortistatin, enkephalin, endothelin, erythropoietin, galanin, gastrin, ghrelin, glucagon, gonadotropin-releasing hormone, growth horming, hepcidin, gonadotropin, lactogen, inhibin, insulin, somatomedin, leptin, lipotropin, motilin, orexin, osteocalcin, parathyroid hormone, prolactin, relaxin, renin, secretinin, somatostatin, thrombopoietin, thyrotropin, guanylin, uroguanylin, serotonin, dopamine, oxytocin, endorphin, a steroid, testosterone, estrogen, dehydroepiandrosterone, androstenedione, dihydrotestosterone, aldosterone, estradiol, estrone, estriol, progesterone, calcitriol, calcidiol, sodium, potassium, magnesium, manganese, selenium, copper, chromium, fluoride, chloride, lithium, beryllium, calcium, bromide, iodide, acetone, methanol, ethanol, or any combination thereof.

In some embodiments, the diagnostic test comprises obtaining biological samples from the treatment set of subjects and the reference set of subjects, and assaying the biological samples to determine the treatment outcomes.

In some aspects, the present disclosure describes a non-transitory computer readable medium comprising machine-executable code that, upon execution by one or more computer processors, implements a method for identifying a clinical intervention for a subject, the method comprising: obtaining a treatment set of subjects and a reference set of subjects, wherein the treatment set of subjects receives the clinical intervention, and wherein the reference set of subjects does not receive the clinical intervention; obtaining treatment outcomes for the treatment set of subjects and the reference set of subjects; performing a set of pairwise comparisons between a first subject selected from the treatment set of subjects and a second subject selected from the reference set of subjects, at least in part by comparing the treatment outcomes between the first subject and the second subject; and identifying the clinical intervention for the subject, based at least in part on the set of pairwise comparisons.

### Computer systems

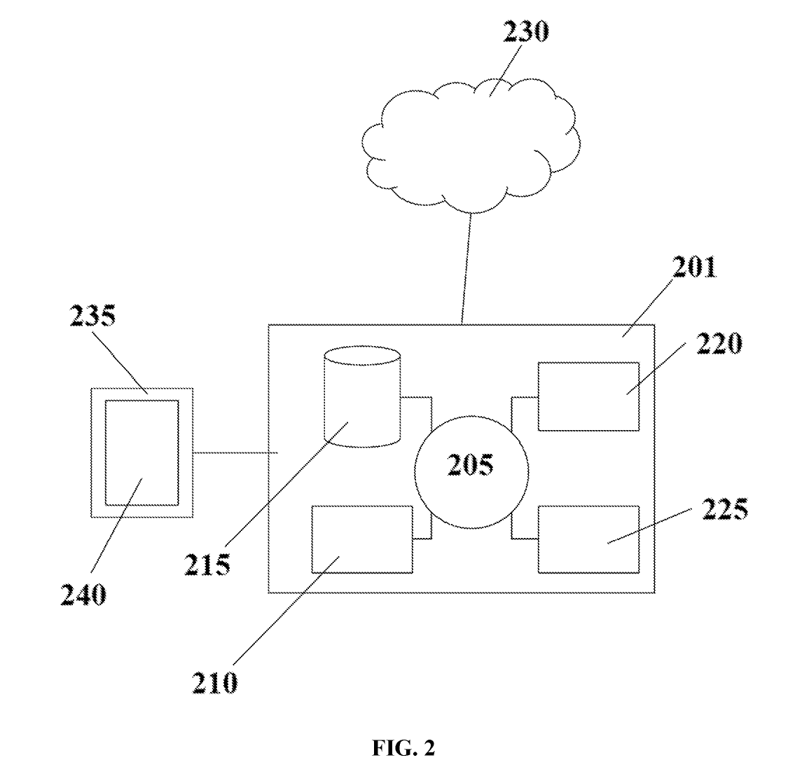

The present disclosure provides computer systems that are programmed to implement methods of the disclosure. FIG. 2 shows a computer system 201 that is programmed or otherwise configured to, for example, identify a net treatment benefit of a clinical intervention for a subject, obtain datasets and treatment outcomes, receive user input of a prioritization function, and perform pairwise comparisons of treatment outcomes between subjects.

The computer system 201 may regulate various aspects of analysis, calculation, and generation of the present disclosure, such as, for example, identifying a net treatment benefit of a clinical intervention for a subject, obtaining datasets and treatment outcomes, receiving user input of a prioritization function, and performing pairwise comparisons of treatment outcomes between subjects. The computer system 201 may be an electronic device of a user or a computer system that is remotely located with respect to the electronic device. The electronic device may be a mobile electronic device.

The computer system 201 includes a central processing unit (CPU, also “processor” and “computer processor” herein) 205, which may be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 may be a data storage unit (or data repository) for storing data. The computer system 201 may be operatively coupled to a computer network (“network”) 230 with the aid of the communication interface 220. The network 230 may be the Internet, an internet and/or extranet, or an intranet and/or extranet that is in communication with the Internet.

The network 230 in some cases is a telecommunication and/or data network. The network 230 may include one or more computer servers, which may enable distributed computing, such as cloud computing. For example, one or more computer servers may enable cloud computing over the network 230 ("the cloud") to perform various aspects of analysis, calculation, and generation of the present disclosure, such as, for example, identifying a net treatment benefit of a clinical intervention for a subject, obtaining datasets and treatment outcomes, receiving user input of a prioritization function, and performing pairwise comparisons of treatment outcomes between subjects. Such cloud computing may be provided by cloud computing platforms such as, for example, Amazon Web Services (AWS), Microsoft Azure, Google Cloud Platform, and IBM cloud. The network 230, in some cases with the aid of the computer system 201, may implement a peer-to-peer network, which may enable devices coupled to the computer system 201 to behave as a client or a server.

The CPU 205 may comprise one or more computer processors and/or one or more graphics processing units (GPUs). The CPU 205 may execute a sequence of machine-readable instructions, which may be embodied in a program or software. The instructions may be stored in a memory location, such as the memory 210. The instructions may be directed to the CPU 205, which may subsequently program or otherwise configure the CPU 205 to implement methods of the present disclosure. Examples of operations performed by the CPU 205 may include fetch, decode, execute, and writeback.

The CPU 205 may be part of a circuit, such as an integrated circuit. One or more other components of the system 201 may be included in the circuit. In some cases, the circuit is an application specific integrated circuit (ASIC).

The storage unit 215 may store files, such as drivers, libraries and saved programs. The storage unit 215 may store user data, e.g., user preferences and user programs. The computer system 201 in some cases may include one or more additional data storage units that are external to the computer system 201, such as located on a remote server that is in communication with the computer system 201 through an intranet or the Internet.

The computer system 201 may communicate with one or more remote computer systems through the network 230. For instance, the computer system 201 may communicate with a remote computer system of a user. Examples ofremote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 201 via the network 230.

Methods as described herein may be implemented by way of machine (e.g., computer processor) executable code stored on an electronic storage location of the computer system 201, such as, for example, on the memory 210 or electronic storage unit 215. The machine executable or machine readable code may be provided in the form of software. During use, the code may be executed by the processor 205. In some cases, the code may be retrieved from the storage unit 215 and stored on the memory 210 for ready access by the processor 205. In some situations, the electronic storage unit 215 may be precluded, and machine-executable instructions are stored on memory 210.

The code may be pre-compiled and configured for use with a machine having a processer adapted to execute the code, or may be compiled during runtime. The code may be supplied in a programming language that may be selected to enable the code to execute in a pre-compiled or as compiled fashion.

Aspects of the systems and methods provided herein, such as the computer system 201, may be embodied in programming. Various aspects of the technology may be thought of as “products” or “articles of manufacture” typically in the form of machine (or processor) executable code and/or associated data that is carried on or embodied in a type of machine readable medium. Machine executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 may include or be in communication with an electronic display 235 that comprises a user interface (UI) 240 for providing, for example, a clinical intervention for a subject. Examples of UIs include, without limitation, a graphical user interface (GUI) and webbased user interface.

Methods and systems of the present disclosure may be implemented by way of one or more algorithms. An algorithm may be implemented by way of software upon execution by the central processing unit 205. The algorithm can, for example, identify a net treatment benefit of a clinical intervention for a subject, obtain datasets and treatment outcomes, receive user input of a prioritization function, and perform pairwise comparisons of treatment outcomes between subjects.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1

This example demonstrates a method for identifying a net treatment benefit of a clinical intervention for a subject.

A patient and a clinician collaborate on selecting a treatment plan that takes into consideration the patient's preferences and needs, using a clinical trial dataset comprising a plurality of treatment plans each associated with a plurality of treatment outcomes. The plurality of treatment outcomes may include efficacy, toxicity, quality of life, or cost-related information. The patient and the clinician rank each treatment outcome from most important (e.g., 1) to the least important (N, where N is the number of treatment outcomes).

The clinical trial dataset may comprise data for various treatments, for example an experimental drug treatment, a chemotherapy, a radiotherapy, an immunotherapy, or a surgical operation. In some embodiments, the clinical dataset may comprise combination treatments of various treatments. The size of the clinical dataset may vary, anywhere from at least about 10, 50, 100, 1000, or 10000 subjects.

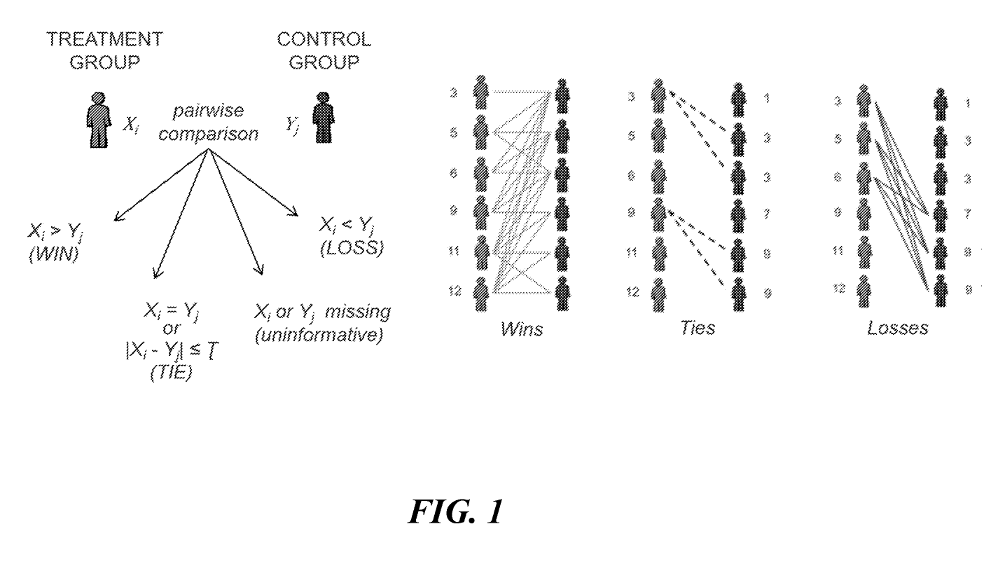

Once the plurality of treatment outcomes is ranked, the following algorithm is executed, as illustrated in FIG. 1:
1. Pairs are formed for the trial data between a first set of patients of the control arm and a second set of patients of the experimental arm. In some embodiments, all possible pairwise comparisons between the first and second sets of patients are analyzed. In some embodiments, the trial data comprises treatment outcomes for an experimental drug treatment, an approved drug treatment, a medical device intervention, a surgical intervention, radiotherapy, an immunotherapy, a digital health intervention, or a behavioral intervention data for the second set of patients. In some embodiments, the treatment outcomes comprise discrete or continuous variables that represent an event-free survival time, progression-free survival time, overall survival time, efficacy, safety, quality of life, or a biomarker level associated with a treatment. In some embodiments, the first set of patients are randomly selected from the control arm and the second set of patients are randomly selected from the experimental arm.
2. Each pair is compared on the first priority:
   a. If the outcome is more favorable for the patient in the experimental arm, the pair is declared a “win”.
   b. If the outcome is more favorable for the patient in the control arm, the pair is declared a “loss”.
   c. If no patient is favored because the outcomes are either equal, or not different enough for proclaiming a clinically relevant winner, the pair is declared a “tie”.
   d. Alternatively, declaring the pair as a “win”, “loss”, or “tie” may be based on the determining a difference in the treatment outcomes between the first subject and the second subject. The pairwise comparison may be characterized as a win, a loss, or a tie based at least in part on the difference in the treatment outcomes being a positive difference greater than a threshold, a negative difference greater than a threshold, or a difference less than a threshold, respectively.
   e. In some embodiments, the likelihood that the patient in the experimental arm has a better treatment outcome than the second subject is also determined.
3. Pairs that are ties on the first priority, are subsequently compared on the second priority in an attempt to classify them as wins, losses or ties again.
4. This recursive classification is operated until either all pairs are classified as wins or losses, or all outcomes have been investigated following the choice of priorities.
5. In some embodiments, the method further comprises determining the net treatment benefit of a clinical intervention for a patient, based at least in part on the set of pairwise comparisons performed. In some embodiments, comprises administering the clinical intervention for the patient based on determination of the net treatment benefit.

Wins and losses are then aggregated over all pairs, and averaged in a single value that estimates the net benefit. The net benefit represents the probability that a random patient in the experimental arm has a more favorable outcome in the trial than a random patient in the control arm, minus the probability of the opposite situation.

The net benefit value may comprise a value between -1 and 1, and it is easy for a clinician or a patient to interpret: a value greater than 0 may signify that the experimental arm is preferred (based on the choice of priorities), while a value less than 0 may signify the opposite conclusion.

Some advantages of this method are that (1) the number of outcomes is virtually unlimited, (2) the outcomes may be of different nature and be arbitrarily complex, (3) clinical relevance is further introduced via thresholds for declaring wins and losses, (4) prioritizing outcomes is easier than assigning weights to them, (5) sensitivity analyses may be carried out, including patient-centric choices of priorities, (6) communication around the net benefit (e.g., between a clinician to a patient, or clinician to clinician) may be much easier than with alternative treatment effect measures that also consider multiple outcomes.

This method may allow patients and clinicians to elicit among the outcomes of the trial, an order of importance that is most relevant to them. Patients may be placed at the center of their medical decisions by rigorously incorporating the patient's preferences, needs, and wants, may be incorporated into a treatment plan. This method has an easy to understand graphical nature which may guide researchers, patients, and clinicians in the analysis of clinical trial results to help them make the best-suited medical decisions.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A computer-implemented method of increasing computational efficiency of analyzing clinical intervention data while considering patient treatment outcome preferences, the method comprising:
generating, by a computing system, a graphical user interface to present a plurality of treatment outcomes;
receiving, by the computing system via the graphical user interface, a ranked priority of the plurality of treatment outcomes from a patient;
forming, by the computing system, a set of pairs from an experimental set of subjects that had been administered a clinical intervention and a control set of subjects that had not been administered the clinical intervention, wherein each pair of the set of pairs includes a first subject from the experimental set of subjects and a second subject from the control set of subjects;
determining, by the computing system, a highest-ranked treatment outcome from the plurality of treatment outcomes based on the ranked priority;
conducting, by the computing system, pairwise comparisons based on the highest-ranked treatment outcome for each pair of the set of pairs to assign each pair a win label, a loss label, or a tie label, wherein at least one but fewer than all of the pairs are assigned the tie label;
determining, by the computing system, a next-highest ranked treatment outcome from the plurality of treatment outcomes based on the ranked priority;
for each pair assigned the tie label but not pairs assigned the win label or the loss label, conducting, by the computing system, a pairwise comparison based on the next-highest ranked treatment outcome to attempt to replace the tie label with the win label or the loss label;
repeating, by the computing system, the determining of the next-highest ranked treatment outcome and the conducting of the pairwise comparison based on the next-highest ranked treatment outcome for each pair assigned the tie label to attempt to replace the tie label with the win label or the loss label until either all of the pairs of the set of pairs have been assigned the win label or the loss label or pairwise comparisons have been conducted for all of the treatment outcomes of the plurality of treatment outcomes;
determining, by the computing system, a net treatment benefit for the clinical intervention based on a number of pairs of the set of pairs assigned the win label and a number of pairs of the set of pairs assigned the loss label; and
in response to determining that the net treatment benefit indicates that the clinical intervention should be administered, causing, by the computing system, the clinical intervention to be administered to the patient.

2. The computer-implemented method of claim 1, wherein forming the set of pairs from the experimental set of subjects that had been administered the clinical intervention and the control set of subjects that had not been administered the clinical intervention includes randomly selecting the first subject from the experimental set of subjects and randomly selecting the second subject from the control set of subjects.

3. The computer-implemented method of claim 1, wherein conducting pairwise comparisons based on the highest-ranked treatment outcome for each pair of the set of pairs to assign each pair a win label, a loss label, or a tie label includes:
determining a difference between the highest-ranked treatment outcome for the first subject and the highest-ranked treatment outcome for the second subject.

4. The computer-implemented method of claim 3, wherein conducting pairwise comparisons based on the highest-ranked treatment outcome for each pair of the set of pairs to assign each pair a win label, a loss label, or a tie label further includes:
assigning the pair the win label in response to determining that the difference is greater than a positive threshold;
assigning the pair the loss label in response to determining that the difference is less than a negative threshold; and
assigning the pair the tie label in response to determining that the difference is between the positive threshold and the negative threshold.

5. The computer-implemented method of claim 1, wherein the ranked priority is received as an ordering, a ranking, a set of weights, or a non-transitive ordering for the plurality of treatment outcomes.

6. The computer-implemented method of claim 1, wherein conducting pairwise comparisons based on the highest-ranked treatment outcome for each pair of the set of pairs to assign each pair a win label, a loss label, or a tie label includes subtracting a measured harm of the clinical intervention from a measured benefit of the clinical intervention.

7. The computer-implemented method of claim 1, wherein the clinical intervention includes one or more of a medication, a cell-based therapy, a gene therapy, a drug treatment, a medical device, a surgical intervention, a chemotherapy, a radiotherapy, radioisotopic/nuclear therapy, physical therapy, occupational therapy, phonoaudiological therapy, a rehabilitation intervention, a psychological intervention, an immunotherapy, a digital health intervention, and a behavioral intervention.

8. The computer-implemented method of claim 1, wherein the treatment outcomes are measured by discrete variables, continuous variables, ordinal variables, or time-to-event variables.

9. The computer-implemented method of claim 1, wherein the plurality of treatment outcomes include two or more of an event-free survival time, a progression-free survival time, an overall survival time, another time to event, an efficacy, a safety, a quality of life, a functional score, a performance score, a toxicity grade, a behavioral score, a composite score, an index score, or a biomarker.

10. The computer-implemented method of claim 9, wherein the biomarker is a chemical biomarker, a genomic biomarker, an epigenomic biomarker, a gene expression biomarker, a protein biomarker, a metabolite biomarker, or a clinical test result corresponding to a disease.

11. A computing system, comprising:
one or more processors; and
a non-transitory computer-readable storage medium having computer-executable instructions stored thereon that, in response to execution by the one or more processors, cause the computing systems to perform actions for increasing computational efficiency of analyzing clinical intervention data while considering patient treatment outcome preferences, the actions comprising:
generating, by the computing system, a graphical user interface to present a plurality of treatment outcomes;
receiving, by the computing system via the graphical user interface, a ranked priority of the plurality of treatment outcomes from a patient;
forming, by the computing system, a set of pairs from an experimental set of subjects that had been administered a clinical intervention and a control set of subjects that had not been administered the clinical intervention, wherein each pair of the set of pairs includes a first subject from the experimental set of subjects and a second subject from the control set of subjects;
determining, by the computing system, a highest-ranked treatment outcome from the plurality of treatment outcomes based on the ranked priority;
conducting, by the computing system, pairwise comparisons based on the highest-ranked treatment outcome for each pair of the set of pairs to assign each pair a win label, a loss label, or a tie label, wherein at least one but fewer than all of the pairs are assigned the tie label;
determining, by the computing system, a next-highest ranked treatment outcome from the plurality of treatment outcomes based on the ranked priority;
for each pair assigned the tie label but not pairs assigned the win label or the loss label, conducting, by the computing system, a pairwise comparison based on the next-highest ranked treatment outcome to attempt to replace the tie label with the win label or the loss label;
repeating, by the computing system, the determining of the next-highest ranked treatment outcome and the conducting of the pairwise comparison based on the next-highest ranked treatment outcome for each pair assigned the tie label to attempt to replace the tie label with the win label or the loss label until either all of the pairs of the set of pairs have been assigned the win label or the loss label or pairwise comparisons have been conducted for all of the treatment outcomes of the plurality of treatment outcomes;
determining, by the computing system, a net treatment benefit for the clinical intervention based on a number of pairs of the set of pairs assigned the win label and a number of pairs of the set of pairs assigned the loss label; and
in response to determining that the net treatment benefit indicates that the clinical intervention should be administered, causing, by the computing system, the clinical intervention to be administered to the patient.

12. The system of claim 11, wherein forming the set of pairs from the experimental set of subjects that had been administered the clinical intervention and the control set of subjects that had not been administered the clinical intervention includes randomly selecting the first subject from the experimental set of subjects and randomly selecting the second subject from the control set of subjects.

13. The system of claim 11, wherein conducting pairwise comparisons based on the highest-ranked treatment outcome for each pair of the set of pairs to assign each pair a win label, a loss label, or a tie label includes:
determining a difference between the highest-ranked treatment outcome for the first subject and the highest-ranked treatment outcome for the second subject.

14. The system of claim 13, wherein conducting pairwise comparisons based on the highest-ranked treatment outcome for each pair of the set of pairs to assign each pair a win label, a loss label, or a tie label further includes:
assigning the pair the win label in response to determining that the difference is greater than a positive threshold;
assigning the pair the loss label in response to determining that the difference is less than a negative threshold; and
assigning the pair the tie label in response to determining that the difference is between the positive threshold and the negative threshold.

15. The system of claim 11, wherein the ranked priority is received as an ordering, a ranking, a set of weights, or a non-transitive ordering for the plurality of treatment outcomes.

16. The system of claim 11, wherein conducting pairwise comparisons based on the highest-ranked treatment outcome for each pair of the set of pairs to assign each pair a win label, a loss label, or a tie label includes subtracting a measured harm of the clinical intervention from a measured benefit of the clinical intervention.

17. The system of claim 11, wherein the clinical intervention includes one or more of a medication, a cell-based therapy, a gene therapy, a drug treatment, a medical device, a surgical intervention, a chemotherapy, a radiotherapy, radioisotopic/nuclear therapy, physical therapy, occupational therapy, phonoaudiological therapy, a rehabilitation intervention, a psychological intervention, an immunotherapy, a digital health intervention, and a behavioral intervention.

18. The system of claim 11, wherein the treatment outcomes are measured by discrete variables, continuous variables, ordinal variables, or time-to-event variables.

19. The system of claim 11, wherein the plurality of treatment outcomes include two or more of an event-free survival time, a progression-free survival time, an overall survival time, another time to event, an efficacy, a safety, a quality of life, a functional score, a performance score, a toxicity grade, a behavioral score, a composite score, an index score, or a biomarker.

20. The system of claim 19, wherein the biomarker is a chemical biomarker, a genomic biomarker, an epigenomic biomarker, a gene expression biomarker, a protein biomarker, a metabolite biomarker, or a clinical test result corresponding to a disease.
